(19) United States
(12) Patent Application Publication
(10) Pub. No.: US 2010/0083966 A1
(43) Pub. Date: Apr. 8, 2010

(54) DEVICE FOR MONITORING, CONTROLLING AND/OR REGULATING A GAS COMPOSITION

(76) Inventor: Ulrich Jerichow, Gelnhausen (DE)

Correspondence Address: K.F. ROSS P.C., 5683 RIVERDALE AVENUE, SUITE 203 BOX 900, BRONX, NY, 10471-0900, US

(21) Appl. No.: 12/522,387

(22) PCT Filed: Dec. 28, 2007
(86) PCT No.: PCT/EP2007/011443

§ 371 Date: Sep. 15, 2009

(30) Foreign Application Priority Data
Jan. 12, 2007 (DE) 10 2007 002 614.7

(51) Int. Cl. A61M 16/00

(52) U.S. Cl. 128/204.22

(57) ABSTRACT

The invention relates to a device and a method for monitoring, controlling and/or regulating a gas composition. Said device comprises at least one training and/or rehabilitation unit; a sensor unit comprising a heatable electrochemical solid electrolyte sensor for oxygen concentration determination and another heatable electrochemical solid electrolyte sensor for carbon dioxide concentration determination; a control unit for the sensors; a microcontroller in the control unit for controlling, according to the respiratory flow volume, the heating power of heating elements of the sensors in order to maintain constant sensor temperatures; and a system for enriching and/or depleting air constituents for producing the gas composition, according to determinable nominal values and/or according to the respiratory gas composition determined by the sensor unit, and the determined respiratory flow volume.

The present invention concerns an apparatus for monitoring, controlling and/or regulating a gas composition of a training and/or rehabilitation system.

It is known that altitude training increases the conditional performance of, for example, an athlete. In the process, a corresponding training effect is achieved by longer training segments in high mountainous regions. However, an exactly assessable and controlled increase in performance is not possible for such training at attitudes.

For a precise examination of pulmonary functions, the process of spirometry can be applied. Thus, for example, DE 6 912 241 describes a spirometric respiratory mask for the examination of pulmonary function, particularly during periods of physical stress. Further, according to US 2006/0201507, a spirometer can be used for measuring oxygen intake. However, it is not possible with such systems to simultaneously monitor the oxygen composition and the carbon dioxide composition of the inhaled as well as the exhaled air.

In order to monitor performance data as well as of bodily functions of the endurance training of an athlete more comfortably, integration of the training unit into a system for providing virtual reality is possible. According to DE 20 2004 007 273, a relaxation and/or an adventure unit, particularly a wellness system with a film presentation by a unit showing a film and a film image section is described in closed rooms, here having movement, sound, wind, smell, light and/or other ray generation units and/or sensor units whose activities and operating modes are coordinated with the adventures of the film presentation in terms of time, location and/or in their intensity. This way, in these and similar systems there is no recording and analysis of body functions such as pulmonary functions, so that also no control of the effect or the success of the application is possible. Further with such systems, no specific fitness or training programs can be preformed or any medical applications.

The object of the present invention is to provide a system with which a person can successfully complete, for example, specific fitness or rehabilitation programs, whereby simultaneously, especially the oxygen composition and the carbon dioxide composition of the inhaled air as well as the exhaled air is monitored, the oxygen content of the inhaled air can be changed as needed, as well as the respiratory flow volume.

The object is attained by an apparatus for monitoring, controlling and/or regulating a gas composition, whereby the apparatus has at least

a training and/or rehabilitation unit,

detecting means with a heatable electrochemical solid electrolyte sensor for determining oxygen concentration and an additional heatable electrochemical solid electrolyte sensor for determining a carbon-dioxide concentration,

a controller for the sensors,

a microprocessor in the controller for controlling the heating power of the heaters of the sensors for maintaining constant sensor temperatures, which depends on the flow of respiratory volume, as well as

a system for depleting and/or enriching air components for generating a gas composition by specifying set points that can be specified and/or dependent on the respiratory gas composition determined by the detecting means, as well as the respiratory flow volume that was determined.

A decisive advantage of such an apparatus is the possibility of being able to determine the oxygen concentration and/or the carbon-dioxide concentration as well as the respiratory flow volume separately or allocable to the inhaled air and also to the exhaled air. A further advantage is that in a training method or also in resting condition, the pulmonary function of a person using the apparatus can be monitored exactly. Further, for example, a critically low level of oxygen can be recognized immediately and the training can be correspondingly modified or stopped. Moreover, expensive calibration, such as is required for spirometers, is no longer necessary.

In an advantageous embodiment, the detecting means is located directly on a component through which the inhaled and exhaled air of the person flows. In this way, the detecting means can, for example, be built into a respiratory mask that is worn by a person. This arrangement has the special advantage that it has very little dead volume.

Within the meaning of this invention, the gas composition that is leaving the system for enriching and/or depleting air components or depleted gas composition can be supplied through tubes of the respiratory mask and thus serve as inhalation air or be mixed with the inhalation air.

It is further advantageous when the oxygen sensor contains zirconium oxide with traces of yttrium as electrolyte for selective conveyance of oxygen ions between two electrodes as well as a support and a heater and the carbon-dioxide sensor contains an electrolyte of a super-fast sodium ion conductor, two electrodes, a support and a heater (1). The mentioned super-fast sodium ion conductor, also called NASICON, can be described by the formula Na₃₋ₓZr₂(PO₄)₁₊ₓ(SiO₄)₂₋ₓ) (2). Sensors of this type have the advantage that they are particularly small and light and can also be manufactured inexpensively. Thus, for sensors of this type, dimensions of, for example 20×3.5×0.5 mm can be achieved (1). Such miniaturized sensors are particularly suitable for installation into a breathing mask.

For measurement of the oxygen saturation of the blood, it is advantageous when an ear clip is integrated into the apparatus. Moreover, the apparatus can contain an ear clip for measuring the pulse rate of the user. Thus, extensive performance data can thus be captured with the apparatus, as well as additional medical parameters of the user such as, for example, the heart rate. The performance data obtained can be advantageously charted on a connected Personal Digital Assistant (PDA).

The training and/or rehabilitation unit can, for example, be a rowing machine or a bicycle or a treadmill.

Advantageous embodiments of the system for enriching and/or depleting air components can contain one or several filters for enriching or depleting gas components, preferably for the depletion of oxygen. Thus, the possibility for a depletion of oxygen can be particularly advantageous for the simulation of altitude training. Of course, the system can also be designed as desired for the depletion or enrichment of air components. Thus, for example, an oxygen concentrator can be present with a membrane filter, the gas composition that is leaving the system with enriched or depleted oxygen being used as needed.

Further, the apparatus can, in advantageous design, comprise means for two-dimensional and three-dimensional visual display, at least one acoustic output and/or recording means and means for the generation of wind, temperature or smell. Further, the apparatus can contain means for simulating the sense of touch and/or means for changing the composition of respiratory air. Further, it is advantageous when the components of the training and/or rehabilitation unit, the system for depleting and/or enriching air components, the detecting means and the controller for the sensors are connected with each other by a computer system, as well as controlled and/or read by such a computer system. In the method, the computer system can consist of at least one controlling computer with a user interface.

In an advantageous variant of an embodiment of the apparatus, a network computer is connected to the controlling computer for image calculation for the right and left eye. The signals generated thereby can be forwarded to a helmet that is worn on the head of the user with LCDs for generating a virtual environment (Head Mounted Display HMD). Alternatively, the generated signals can also be used for stereo production for generating a three dimensional image on a screen. Further, it is advantageous when the controlling computer is equipped with one or more input devices with at least six degrees of freedom for determining position and orientation, and the input devices are selectively equipped with one or more buttons. Further, it is advantageous that, for example, isometric, isotonic and/or elastic input devices are connected to the controlling computer, the movement of sight, movement of the body, movement of the head and/or position determination can be captured or for instance take place via these input devices. In a further advantageous design, the input devices can capture gestures, mimic and/or speech. Thus, a combination of physical and mental stimuli is made possible and an application of aroma or altitude training can be performed in a virtual three-dimensional environment.

In a particularly preferred variant of an embodiment, a head tracker is used, for example, as an input device that can also be mounted on the helmet with LCDs worn on the head of the user for generating the virtual environment (Head Mounted Display HMD). Further, it is advantageous when the visual image unit shows a stationary image, a moving or non-moving object, a computer graphic and/or two-dimensional and/or three-dimensional moving images or films. For this, conventional monitors for the two-dimensional display can also be used.

In an advantageous embodiment, the visual display unit can render an image with a viewing angle of 0 to 179°, or for use of the system in the areas of fitness, wellness or medicine also an image with a viewing angle of 180° or more than 180°, moving and/or still real images that have previously been recorded by the user being displayable as well.

The acoustic output unit can, for example, play back musical instruments, human voices, environmental noises such as animal sounds, wind, rain, waterfalls, thunder and/or noises of motor vehicles, shots, pumps, explosions and/or construction work. It is particularly advantageous when wind, temperature, smell and/or humidity can be coordinated with the situation displayed in virtual reality.

Further, it is advantageous when instructions and/or pointers can be given to the user by a communication unit of the apparatus and the user can contact a person starting the apparatus via a communication unit. In an advantageous further development of the system, even more precise blood analyses can be performed prior to, during and/or after use by drawing blood samples. For example, with the aid of a cell analyzer that is connected with the is computer system, preferably an apparatus for flow cytometry, the composition of blood cells can be determined exactly. By using a specific antibody, preferably coupled with a fluorescent pigment, an analysis of surface markers on cells is possible.

Within the meaning of this invention is an additional method for simultaneous monitoring, controlling and/or regulating a gas composition, whereby
a person uses a training unit and/or a rehabilitation unit,
oxygen concentration is determined by detecting means with the aid of a heatable electrochemical solid electrolyte sensor and a carbon-dioxide concentration is determined with the help of an additional heatable electrochemical solid electrolyte sensor,
depending on the respiratory air flow volume of the person, controlling the heating power of the heaters of the sensors for maintaining constant sensor temperatures with the help of a microprocessor in the sensor controller, as well as
depending on predetermined set points and/or on the respiratory air gas composition, as well as on the respiratory air flow volume of the person, a system for generating the gas composition depletes and/or enriches air components.

This method in accordance with the invention, by using the apparatus described above, can be performed in one or more of the mentioned embodiments. Particularly advantageous, the determination of the oxygen concentration of the respiratory air takes place by measuring the electric current flowing from the cathode to the anode through the electrolyte in the oxygen sensor at constant voltage, with a linear connection between the resulting electric current and the oxygen concentration. Further, it is advantageous when the carbon-dioxide concentration is determined by the logarithmic relationship between the voltage between the electrodes of the carbon-dioxide sensor and the carbon-dioxide concentration. Further, it is advantageous that the respiratory air volume is determined by the heating power of the heaters that is required for maintaining the constant sensor temperature, which is controlled by the microprocessor.

The determination of the total flow rate of the respiratory air can take place with the sensor element by utilizing thin-layer anemometry. Further, the direction of flow of the breathable gas can either be determined by using the measured oxygen concentration gradient and/or the carbon-dioxide concentration gradient or the temperature profile on the sensor. The procedure in accordance with the invention has the advantage that simultaneously the volume stream, the direction of flow and thus the oxygen composition and the carbon dioxide composition of the inhaled air, as well as of the exhaled air can be monitored breath by breath. Thus the oxygen concentration and the carbon-dioxide concentration can clearly be associated with the inhalation air and the exhalation air. This way, it is particularly advantageous when the oxygen content of the inhalation air is changed as needed.

Further, it is advantageous when a critically low level of oxygen is detected in the oxygen concentration of the exhalation air. In an advantageous embodiment of the method in accordance with the invention, when the oxygen content of the inhalation air is critically low, it is increased or the program is stopped. As per the method according to the invention, the user can change the oxygen content within a few seconds from a low level of between 9% by volume up to a high level of up to 100% by volume and the reverse, so that particularly advantageously, an adjustment to a range of 16% by volume up to 21% by volume can take place. Thus, the apparatus in accordance with the invention can be used for increasing endurance performance, preferably by means of simulated altitude training. In this application, it is particularly advantageous that the oxygen concentration can be changed within a few seconds from a low level of 9% by volume to a high level of up to 100% by volume and the reverse, so that a particularly preferred adjustment to a range of 16% by volume up to 21% by volume takes place.

Further, it is advantageous that a computer program with program code for the execution of all procedural steps in accordance with the invention that are mentioned above is executed when the program is run on a computer. This way, it is advantageous when the computer program with the program code for the execution of all procedural steps that are mentioned above is stored on machine-readable media when the program is executed in a computer.

By using the apparatus in accordance with the invention and/or the procedure according to the invention, top athletes and performance athletes, for example, can optimally prepare with altitude training units in a virtually real environment for impending competitions. The virtually real training in conditions with low oxygen targets an increase in personal performance and the individual conditioning level of professional and amateur athletes. This way, specific costs and time-consuming flights and stays in high mountain regions can be avoided. Further, significantly increased efficiency of training is possible as the system is available for 24 hours and is easily reachable logistically.

In the area of rehabilitation or wellness, this system could, in a virtual three-dimensional environment, combine, for example, an aroma application with passive altitude training and oxygen therapy. In such an environment, such a combination of relaxation and improvement of personal physical performance, as well as strengthening of the immune system could be achieved.

In the area of medicine, the system can be used for an aroma application, altitude training and/or oxygen therapy in a three-dimensional environment, so that the four senses—seeing, feeling, smelling and hearing—are stimulated. As a result of the mobilization of the body's defense systems that are mobilized thereby, use by persons with diseases such as, for example, cancer, allergies and metabolic diseases is conceivable.

Further, particularly the technology of the three-dimensional image offers the possibility of positively influencing the course of specific physical diseases such as anxiety in autoimmune system diseases, as a result of the effect of images and sounds.

### LITERATURE

R. Baumann^{1,2}, S. Fasoulas¹, M. Gläser¹, C. Gritzner¹, F. Hammer², J. Heisig¹, R. Kahle¹, T. Kirschke¹, T. Schmiel¹, M. Völkel². Solid State Electrolyte Sensors for the Determination of Oxygen, Carbon Dioxide, and Total Flow Rates Associated to Respiration in Human Subjects. ¹Institute for Aerospace Engineering, Technical University Dresden, 01062 Dresden, Germany²ESCUBE GmbH, Nobelstr. 15, 70569 Stuttgart, Germany (PRO2-FR-Exec-Sum-05-02-10 Executive Summary to the ESTEC Contract number 15450/01/NL/JS CCN 1+2)
West, A. R., Basics of Solid Body Chemistry [Grundlage der Festkorperchemie], publisher: Verlag Chemie, Weinheim (1992).

1. An apparatus for monitoring, controlling and/or regulating a gas composition, the apparatus comprising:
a training and/or rehabilitation unit,
detecting means with a heatable electrochemical solid electrolyte sensor for determining the concentration of oxygen and an additional heatable electrochemical solid electrolyte sensor for determining a carbon-dioxide concentration,
a controller for the sensors,
a microprocessor in the controller for controlling the heating power of heaters of the sensors for maintaining constant sensor temperatures that are dependent on the respiratory flow volume, and
a system for depleting and/or enriching air components for generating a gas composition with predetermined set points and/or dependent on the respiratory gas composition determined by the detecting means, as well as the detected respiratory flow volume.

2. The apparatus according to claim 1 wherein the detecting means is provided directly in a component through which the inhalation and exhalation air of a person flows.

3. (canceled)

4. The apparatus according to claim 1 wherein the oxygen sensor for selective conveyance of oxygen ions contains zirconium oxide with traces of yttrium as electrolyte between two electrodes, as well as a support and a heater.

5. The apparatus according to claim 1 wherein the carbon-dioxide sensor contains an electrolyte of a super-fast sodium ion conductor, two electrodes, a support and a heater.

6-7. (canceled)

8. The apparatus according to claim 1 wherein performance data are captured, and additional medical parameters of the user such as, for example, the heart rate are recorded.

9-11. (canceled)

12. The apparatus according to claim 1 further comprising
means for two-dimensional and/or three-dimensional visual display, at least one acoustic output and/or recording means and means for the generation of wind, temperature and/or smell.

13. The apparatus according to claim 1 further comprising
means for the stimulation of the sense of touch and/or means for changing the composition of the respiratory air.

14. The apparatus according to claim 1 wherein the components of the training and/or rehabilitation unit of the system for depleting and/or enriching air components, the detecting means and the controller for the sensors are connected with each other, controlled by, and or read by a computer system.

15-18. (canceled)

19. The apparatus according to claim 14 wherein the controlling computer is equipped with one or more input devices with at least six degrees of freedom for determination of position and orientation, and the input devices are selectively equipped with one or more buttons.

20-23. (canceled)

24. The apparatus according to claim 12 wherein the visual imaging unit shows a still image, a moving or an non-moving object, a computer graphic and/or two-dimensional and/or three-dimensional moving images or films.

25-27. (canceled)

28. The apparatus according to claim 12 wherein the acoustic output unit plays back musical instruments, human voices, environmental sounds such as animal sounds, wind, rain, waterfalls, thunder and/or noises of vehicle engines, shots, pumps, explosions or construction work.

29-31. (canceled)

32. The apparatus according to claim 14 wherein a cell analyzer is connected with the computer system, preferably an apparatus for flow cytometry.

33. A method for simultaneous monitoring, controlling and/or regulating of a gas composition, the method comprising the steps of:
a person using a training unit or a rehabilitation unit,
detecting means determine oxygen concentration with a heatable electrochemical solid electrolyte sensor and an additional heatable electrochemical solid electrolyte sensor determines a carbon-dioxide concentration,
a microprocessor in the detecting means controls heating power of the heaters of the sensors to maintain constant sensor temperatures dependent on the respiratory volume flow of a person, as well as
a system depletes and/or enriches components of the air depending on predetermined set points and/or on the respiratory gas composition determined by the detecting means, as well as the determined respiratory flow volume of the person.

34-43. (canceled)

44. Use of the apparatus according to claim 1 for increasing endurance performance for altitude training.

45. (canceled)

46. A computer program with program code for the execution of all processing steps claim 33 when the program is executed in a computer.

47. (canceled)
